(19) United States
(12) Patent Application Publication
(10) Pub. No.: US 2025/0213105 A1
(43) Pub. Date: Jul. 3, 2025

(54) ENDOSCOPE ASSEMBLY, AND OPERATION-ASSISTING DEVICE FOR TOOL FOR TREATMENT OR SURGERY

(71) Applicant: TOHOKU UNIVERSITY, Miyagi (JP)

(72) Inventors: Daisuke YAMAUCHI, Miyagi (JP); Yoichi HAGA, Miyagi (JP); Noriko TSURUOKA, Miyagi (JP); Yukio KATORI, Miyagi (JP); Tetsuaki KAWASE, Miyagi (JP); Ken YAGIHASHI, Miyagi (JP)

(21) Appl. No.: 18/850,324

(22) PCT Filed: Mar. 10, 2023
(86) PCT No.: PCT/JP2023/009339

§ 371 Date: Sep. 24, 2024

(30) Foreign Application Priority Data
Mar. 25, 2022 (JP) 2022-049612

(51) Int. Cl. A61B 1/227; A61B 1/04; A61F 11/20

(52) U.S. Cl. CPC A61B 1/227; A61B 1/042; A61F 11/20

(57) ABSTRACT

An endoscope assembly (10) for operation assistance for a treatment or surgery tool. The endoscope assembly includes: an imaging element (11); a shaft (13) that is configured to accommodate the imaging element or is connected to the imaging element; a handle unit (14) extending from the shaft while expanding; and a mounting unit (15) attached to the shaft to mount a treatment or surgery tool.

### TECHNICAL FIELD

The present invention relates to an endoscope assembly for operation assistance for a treatment or surgery tool.

### BACKGROUND ART

As a related-art ear surgery, there has been performed a microsurgery (FIG. 1(A), 1953-, Wullstein) in which a bone drilling region 102 is provided in an ear bone 101 so that a diseased part 100 can be visually recognized, and a surgery is performed while the diseased part 100 is irradiated with light 104 from a microscope 103 and is observed under the microscope 103 (a reference numeral 105 denotes an Auricle). This method has the following problems. Specifically, the method is invasive because the bone drilling region 102 is formed, and a blind spot is liable to occur when the bone drilling region 102 is made smaller in order to reduce invasiveness.

An endoscopic surgery having low invasiveness as its major advantage has been dramatically progressing recently. A transcanal endoscopic ear surgery (FIG. 1(B), 1998-, Tarabichi) in which an endoscope 107 is inserted into an external ear canal 106, and a surgery is performed while a diseased part 109 is irradiated from a distal end 108 of the endoscope 107 and is observed under the endoscope 107 has only recently been made possible as an endoscopic surgery.

In recent years, Yamauchi et al., the inventors of the present application, have developed also an underwater endoscopic ear surgery (FIG. 1(C), 2014-, Yamauchi, Patent Literature 1) in which a periphery of a diseased part is filled with water or an aqueous solution 110, and a surgery is performed while the diseased part is observed with an endoscope 111 under water.

However, an endoscopic surgery is, due to an extremely small accessible space, typically a one-hand surgery in which a surgical operation is performed with a dominant hand with an endoscope 112 held in one hand (FIG. 2(A)). A one-hand surgery is advantageous in its good field of view, but is disadvantageous in that training is required for procedures of surgical operations and thus a learning curve is gentle. Further, ear surgical operations include aspiration, grasping, traction, dissection, division, facial nerve stimulation, and extirpation of a lesion site with electrocoagulation hemostasis, and an eardrum or sound conduction is reconstructed by the above-mentioned operations, but it is not easy to perform all the operations with one hand.

A possible solution is to fix the endoscope 112 with a commercially-available endoscope holder 114 and perform a two-hand surgery in which a surgical operation is performed with both hands (FIG. 2(B)). However, when the endoscope 112 is fixed, the field of view is fixed, and hence the endoscope cannot be freely moved. Further, it is difficult to grasp the three-dimensionality. Moreover, there are problems of difficulties in observation due to an operating field varying as a patient moves, expensiveness of the holder, and the like.

Another possible solution is a three-hand surgery (FIG. 2(C)) in which an assistant holds the endoscope 112, and two surgeons perform a surgical operation with both hands. However, it is difficult to acquire skills therefor, and teamwork is required. Hence, such a surgery cannot always be easily performed. Further, in a three-hand surgery, a hand interferes at an extremely narrow portion in an external ear canal, and thus a three-hand surgery cannot be performed in all steps in a surgery.

### CITATION LIST

### Patent Literature

PTL 1: WO 2017/171085 A1

### SUMMARY OF INVENTION

### Technical Problem

An object of the present invention is to provide an endoscope that enables operations of an elongated operating tool such as a treatment or surgery tool with a hand holding the endoscope.

### Solution to Problem

The present invention includes embodiments described below.

Item 1. An endoscope assembly for operation assistance for a treatment or surgery tool, the endoscope assembly including:
an imaging element;
a shaft, which is configured to accommodate the imaging element or is connected to the imaging element;
a handle unit extending from the shaft while expanding; and
a mounting unit attached to the shaft, to mount a treatment or surgery tool.

Item 2. The endoscope assembly according to Item 1, wherein the mounting unit is positioned closer to a distal end of the shaft than the handle unit.

Item 3. The endoscope assembly according to Item 1, wherein the treatment or surgery tool is mounted onto the mounting unit.

Item 4. The endoscope assembly according to Item 3, wherein the treatment or surgery tool is a stick-shaped tool including an operating handle.

Item 5. The endoscope assembly according to any one of Items 1 to 4,
wherein the shaft includes a distal shaft portion and a proximal shaft portion, and
wherein the distal shaft portion is rotatable relative to the proximal shaft portion.

Item 6. The endoscope assembly according to Item 5, wherein the distal shaft portion and the proximal shaft portion are coupled by a flexible coupling member.

Item 7. The endoscope assembly according to Item 5, further including a traction member that has a proximal end fixedly attached to the distal shaft portion, and has a distal end extending outward from the proximal shaft portion, wherein the distal shaft portion is bent when the traction member is withdrawn toward a proximal side.

Item 8. The endoscope assembly according to any one of Items 1 to 4,
wherein the shaft includes a distal shaft portion, a proximal shaft portion, and a substantially tubular member that is attached to an outer peripheral surface of the proximal shaft portion and is rotatable relative to the proximal shaft portion, and
wherein the mounting unit for the treatment or surgery tool is attached to the substantially tubular member.

Item 9. The endoscope assembly according to any one of Items 1 to 4,
wherein the shaft includes a distal shaft portion and a proximal shaft portion,
wherein the handle unit is attached onto an outer periphery of the proximal shaft portion along a longitudinal part of the proximal shaft portion,
wherein the endoscope assembly further includes:
a traction member that has a proximal end fixedly attached to the distal shaft portion, extends through an outside or a bore of each of the distal shaft portion and the proximal shaft portion, and terminates on a proximal side with respect to the proximal shaft portion; and
a mechanism extending through the handle unit from an outside of the handle unit to an inside of the handle unit, the mechanism being configured to selectively advance or withdraw the traction member.

Item 10. The endoscope assembly according to any one of Items 1 to 4, wherein the imaging element is accommodated in the shaft.

Item 11. The endoscope assembly according to any one of Items 1 to 4, wherein the imaging element is connected to a proximal end of the shaft.

Item 12. An operation assisting device for a treatment or surgery tool, which is configured to be used in connection to an endoscope including an imagine device, the operation assisting device including:
a shaft configured to permit the imaging device to pick up an image of a subject via a distal end of the shaft,
wherein the shaft includes:
   a handle unit extending from the shaft while expanding; and
   a mounting unit attached to the shaft, to mount a treatment or surgery tool.

### Advantageous Effects of Invention

According to the present invention, a surgeon can perform a surgical operation with both hands while obtaining a good field of view in accordance with a motion of a patient, to thereby improve operability of a surgery.

### BRIEF DESCRIPTION OF DRAWINGS

FIG. 1(A) is an explanatory view of a microsurgery, FIG. 1(B) is an explanatory view of a transcanal endoscopic ear surgery, and FIG. 1(C) is an explanatory view of an underwater endoscopic ear surgery (taken from URL: http://kompas.hosp.keio.ac.jp/sp/contents/medical_info_presentation_201708.html).

FIG. 2(A) is an explanatory view of a one-hand surgery, FIG. 2(B) is an explanatory view of a two-hand surgery, and FIG. 2(C) is an explanatory view of a three-hand surgery (taken from URL: http://kompas.hosp.keio.ac.jp/sp/contents/medical_info_presentation 201708.html).

FIG. 3 is a perspective view of an endoscope assembly according to a first embodiment.

FIG. 4 is a view of application of the endoscope assembly in FIG. 3 to a transcanal endoscopic ear surgery (TEES).

FIG. 5 is a perspective view of an endoscope assembly according to a second embodiment.

FIG. 6 is a schematic view of a state in which the endoscope assembly in FIG. 5 is held with a left hand.

FIG. 7 is an explanatory view of a mounting unit.

FIG. 8 is a perspective view of an endoscope assembly according to a third embodiment.

FIG. 9 is a schematic view for illustrating a process in which a distal shaft portion is being bent relative to a proximal shaft portion while a shaft of the endoscope assembly in FIG. 8 is inserted into an external ear canal.

FIG. 10 is a view of a modification example of the endoscope assembly according to the embodiment in FIG. 8.

FIG. 11 is a view of an example of a fixing member.

FIG. 12 is a perspective view of an endoscope assembly according to a fourth embodiment.

FIG. 13 is a side view of the endoscope assembly in FIG. 12.

FIG. 14(A) and FIG. 14(B) are views of a transmastoid superior semicircular canal closure surgery in which a one-hand operation in FIG. 2(A) is performed.

FIG. 15(A) and FIG. 15(B) are views of a transmastoid superior semicircular canal closure surgery in which a two-hand surgery using the endoscope assembly according to the fourth embodiment is performed.

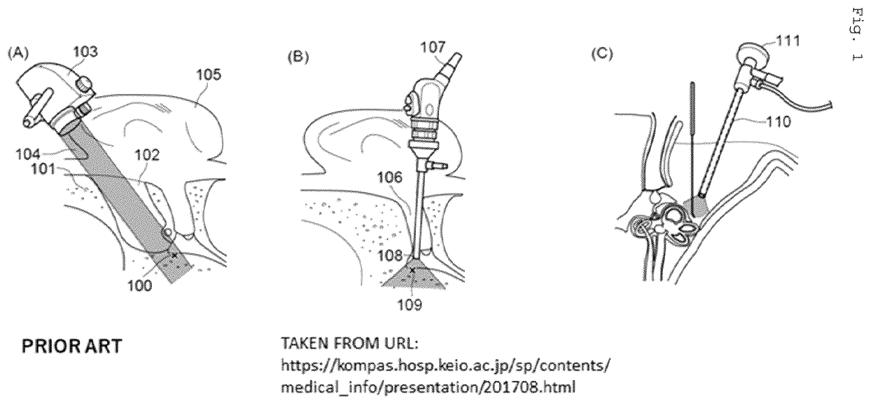

### DESCRIPTION OF EMBODIMENTS

Now, some embodiments of the present invention are described with reference to the drawings.

### First Embodiment

FIG. 3 is a perspective view of an endoscope assembly 10 for operation assistance for a treatment or surgery tool according to a first embodiment. The endoscope assembly 10 includes a complementary metal oxide semiconductor (CMDS) image sensor 11 constituting an imaging element and a substantially cylindrical shaft 13 that accommodates the CMOS image sensor 11.

In the shaft 13, an optical fiber (not shown) extends along a longitudinal direction of the shaft 13, and illuminating light from a light source (not shown) such as an LED is guided from a proximal end to a distal end by the optical fiber (not shown) optically connected to the light source (not shown), to irradiate a subject. Such a configuration of an optical system for an endoscope is known.

At a distal end portion of the shaft 13, an object lens (not shown) is incorporated, and light from the subject passes through the object lens (not shown) from a light-transmitting distal end 13a of the shaft 13, and forms an image in the CMOS image sensor 11. The image of the subject picked up in the CMOS image sensor 11 is transmitted to a signal cable 12 connected to the CMOS image sensor 11 and is subjected to signal processing. The signal cable 12 is connected to a computer, and the image of the subject can be observed with a display device connected to the computer. The signal cable 12 may protrude out of the shaft 13 in a side surface of the shaft 13.

The endoscope assembly 10 further includes a handle unit 14 that extends from the shaft 13 while expanding. The handle unit 14 that extends from the shaft 13 while expanding means the handle unit 14 that extends beyond a diameter and/or a length of the shaft 13. Meanwhile, the handle unit 14 may be removable from the shaft 13 with a screw or the like, which allows the handle unit 14 to be replaced depending on a type of a surgery and a hand size and preference of a surgeon. Specifically, in FIG. 3, a distal end of the handle unit 14 is attached to a proximal end portion of a substantially cylindrical side surface of the shaft 13, and the handle unit 14 extends from a distal end to a proximal end while being inclined with respect to an axis of the shaft 13. The handle unit 14 extends outward along a radial direction of the shaft 13 beyond a diameter of the shaft 13, and extends farther toward a proximal side than the proximal end of the shaft 13. Since the handle unit 14 extends outward along the radial direction of the shaft 13 beyond the diameter of the shaft 13, a space is left on the proximal side with respect to the proximal end of the shaft 13. Thus, the handle 14 is less liable to interfere with a second treatment or surgery tool 18 operated by an opposite hand of an operator.

A material forming the shaft 13 and the handle unit 14 is not limited and may be metal, synthetic resin, a composite material including at least any one thereof, or the like. The synthetic resin includes thermoplastic resin, thermosetting resin, elastomer, and rubber. For example, it is preferred that the shaft 13 and the handle unit 14 be an integral molding of synthetic resin in terms of manufacture and operability.

The endoscope assembly 10 further includes a mounting unit 15 that is attached to the shaft 13, to mount a treatment or surgery tool 16. The mounting unit 15 includes a holding portion 15a for holding or grasping the treatment or surgery tool 16, a fixing portion 15b for fixedly attaching the mounting unit 15 to the side surface of the shaft 13, and a coupling portion 15c coupling the holding portion 15a and the fixing portion 15b.

In this embodiment, the holding portion 15a is an elongated member having a substantially U-shaped section, and holds or grasps a first elongated portion 16b of the treatment or surgery tool 16 in such a manner that the first elongated portion 16b can move back and forth along its longitudinal direction and can be removed from the holding portion 15a. A material forming the holding portion 15a is not limited and may be metal, synthetic resin, a composite material including at least any one thereof, or the like. For example, the holding portion 15a may be made of synthetic resin, and the first elongated portion 16b may be fit into the holding portion 15a by snap-fit using elasticity of the holding portion 15a.

The fixing portion 15b is substantially frustum-shaped in this embodiment, but is not limited to that as long as the fixing portion 15b can be fixed to the side surface of the shaft 13. The fixing of the fixing portion 15b to the side surface of the shaft 13 is achieved by a freely-selected fixedly joining method such as adhesion using an adhesive or welding. A material forming the fixing portion 15b is not limited and may be metal, synthetic resin, a composite material including at least any one thereof, or the like. The synthetic resin includes thermoplastic resin, thermosetting resin, elastomer, and rubber.

It is preferred that the coupling portion 15 be an elastic member such as a spring or a metal leaf spring in particular, or a flexible member, in order to enhance movability and operability of the treatment or surgery tool 16 mounted onto the holding portion 15a. A method of connecting the holding portion 15a and the fixing portion 15b, and the coupling portion 15c is not limited to any particular method, and a freely-selected known method can be used. A material forming the coupling portion 15c is not limited and may be metal, synthetic resin, a composite material including at least any one thereof, or the like. The synthetic resin includes thermoplastic resin, thermosetting resin, elastomer, and rubber.

The treatment or surgery tool 16 includes a distal end portion 16a, the first elongated portion 16b continuously extending from the distal end portion 16a at an angle to the distal end portion 16a, a second elongated portion 16c continuously extending from the distal end portion 16a at an angle to the first elongated portion 16b, and an operating handle 16d attached to a proximal end of the second elongated portion 16c. The operating handle 16d has a diameter larger than those of the first elongated portion 16a and the second elongated portion 16b for an operator (also called a surgeon) to easily grasp. In this embodiment, the operating handle 16d has a substantially spherical shape, but may have any other freely-selected shape that can be easily grasped by the operator. A material forming the operating handle 16d is not limited and may be metal, synthetic resin, a composite material including at least any one thereof, or the like. The synthetic resin includes thermoplastic resin, thermosetting resin, elastomer, and rubber.

In this embodiment, a raspatory is described as an example of the treatment or surgery tool 16, but any other treatment or surgery tool that enables treatment such as dissection, extraction, incision, crushing, and hemostasis may be used. It is preferred that the treatment or surgery tool be a bar-shaped or stick-shaped tool including an elongated portion to be mounted onto the mounting unit 15. Such a treatment or surgery tool is not limited to any particular tool, and examples thereof include a biopsy forceps, a snare, a hemostatic clip, a knife, an ultrasonic instrument (used for tissue incision, tissue dissection, tissue crushing, and/or emulsification of a biological sample, and the like), a radio-frequency energy supply device, a laser device, a perfusion device or an irrigation device that supplies a perfusion liquid during an underwater endoscopic ear surgery, and the like. The distal end portion 16a of the treatment or surgery tool 16 may be removable from the first elongated portion 16b, but the mounting unit 15 is regarded as holding the treatment or surgery tool 16 also in a case in which the mounting unit 15 holds a part of the treatment or surgery tool 16.

The mounting unit 15 is positioned closer to the distal end of the shaft 13 than the handle unit 14. Further, it is preferred that a movable range of the handle unit 14 do not overlap a movable range of the treatment or surgery tool 16 so that the handle unit 14 does not interfere with an operation of the treatment or surgery tool 16 during the operation of the treatment or surgery tool 16 by the operator.

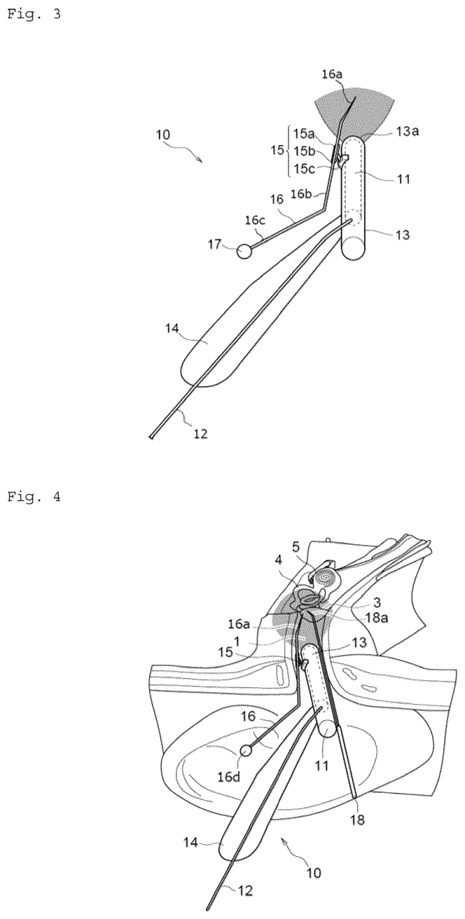

Next, operations of the endoscope assembly 10 are described. First, the treatment or surgery tool 16 is mounted onto the holding portion 15a of the mounting unit 15. Subsequently, the operator grasps an end portion of the shaft 13 of the endoscope assembly 10 and the operating handle 16d of the treatment or surgery tool 16, and inserts the treatment or surgery tool 16 and the shaft 13 into a body cavity of a subject from the distal ends. FIG. 4 is a schematic view of a state in which the endoscope assembly 10 of this embodiment is inserted into an external ear canal 1 in a transcanal endoscopic ear surgery (TEES), as an example. There are illustrated an eardrum 3, semicircular canals 4, and a cochlea 5. A diseased part around the eardrum 3 is irradiated from the light source (not shown) in the endoscope assembly 10, and an image of the diseased part is picked up by the CTOS image sensor 11. When the operator wants to change a position of the diseased part to be imaged, the operator operates the handle unit 14 with fingers (a forefinger, a middle finger, a ring finger, and the like) of the left hand and thus changes the position of the shaft 13, thereby being capable of changing the position of the diseased part to be imaged. Further, the operator can perform treatment such as incision, dissection, and aspiration of the eardrum 3 and the vicinity thereof with the distal end portion 16a of the treatment or surgery tool 16 by grasping and operating the operating handle 16d of the treatment or surgery tool 16, separately from an operation of the handle unit 14. Moreover, the operator can operate another treatment or surgery tool 18 with the right hand and perform treatment, separately from an operation with the left hand. When the treatment of the patient ends, the operator grasps the end portion of the shaft 13 of the endoscope assembly 10 and the operating handle 16d of the treatment or surgery tool 16, withdraws the shaft 13 and the treatment or surgery tool 16, and removes the endoscope assembly 10 from the body cavity of the subject.

Effects produced by the endoscope assembly 10 for operation assistance for a treatment or surgery tool according to the above-mentioned first embodiment are described.
The surgeon can operate the endoscope and the treatment or surgery tool with the same hand.
The surgeon can operate the treatment or surgery tool with a hand holding the endoscope and can operate another treatment or surgery tool with another hand, which enables a two-hand operation by the surgeon. Procedures such as aspiration, grasping, traction, dissection, and division can be performed with two hands. Alternatively, another operation can be also performed with two hands. (For example, at the time of fitting a piston while suppressing dislocation of an incus at a malleus-incus joint in a stapes surgery.)

The endoscope assembly 10 for operation assistance for a treatment or surgery tool according to the above-mentioned first embodiment can be modified as follows.
The CMOS image sensor 11 is used as the imaging element in the first embodiment, but another imaging element may be used.
In FIG. 4, application of the endoscope assembly 10 to a transcanal endoscopic ear surgery (TEES) is illustrated, but the endoscope assembly 10 can be applied also to an underwater endoscopic ear surgery (UWEES) as described in Patent Literature 1.
The endoscope assembly 10 is used with the left hand of the right-handed operator in the above description, but it is possible to use the endoscope assembly 10 with the right hand by rotating the endoscope assembly 10 through 180° about the axis of the shaft 13.

### Second Embodiment

Next, an endoscope assembly 20 for operation assistance for a treatment or surgery tool according to a second embodiment of the present invention is described. Description of members corresponding to those in the first embodiment of the present invention is omitted.

FIG. 5 is a perspective view of the endoscope assembly 20 for operation assistance for a treatment or surgery tool according to the second embodiment. The endoscope assembly 20 includes a CCD camera 21 including a charged coupled device (CCD) constituting an imaging element, and a substantially cylindrical shaft 23 connected to the CCD camera 21. The shaft 23 is connected to the CCD camera 21 at a proximal end of the shaft 23. In this embodiment, the shaft 23 includes a distal shaft portion 23a that is hollow and substantially cylindrical and a proximal shaft portion 23b that is hollow and substantially cylindrical, and the distal shaft portion 23a is rotatable relative to the proximal shaft portion 23b.

In the shaft 23, an optical fiber (not shown) extends along a longitudinal direction of the shaft 23, and illuminating light from a light source (not shown) such as an LED connected to a proximal end of the optical fiber (not shown) is guided from the proximal end to the distal end by the optical fiber (not shown), to irradiate a subject.

At a distal end portion of the shaft 23, a lens (not shown) is incorporated, and light from the subject passes through the object lens (not shown), the optical fiber, an eyepiece such as a gradient index (GRIN) lens from a light-transmitting distal end of the shaft 23, and forms an image in the CCD camera 21. The image of the subject picked up in the CCD camera 21 is transmitted to a signal cable 22 and is subjected to signal processing. The signal cable 22 is connected to a computer, and the image of the subject can be observed with a display device connected to the computer. It is preferred that a field of view be a field of view for 4K or higher.

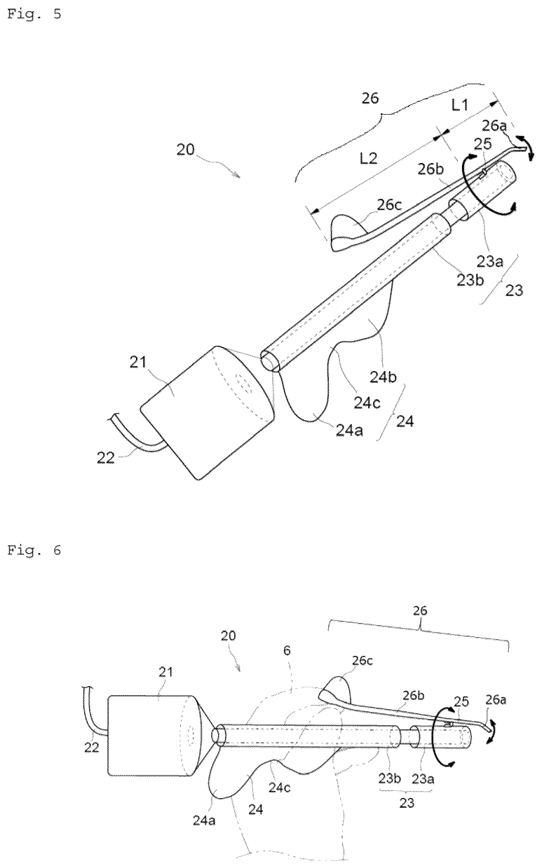

The endoscope assembly 20 further includes a handle unit 24 that extends from the shaft 23 while expanding. Specifically, the handle unit 24 extends from a spot on a circumference of the shaft 23 in a side surface of the shaft 23 along a longitudinal direction of the shaft 23 and outward in a radial direction beyond a diameter of the shaft 23. The handle unit 24 includes two protrusions 24a and 24b having curved surfaces, and a recess 24c is formed between the two protrusions 24a and 24b. In this embodiment, the protrusion 24b protrudes more in a direction perpendicular to a longitudinal axis of the shaft 23, than the protrusion 24a. The handle unit 24 has a continuously curved surface except a junction with the shaft 23 therein, and has a shape that is better fit for the surgeon's hand and reduces fatigue of the surgeon by its ergonomic design. Specifically, as illustrated in FIG. 6, during use of the endoscope assembly 20, the handle unit 24 is grasped by a thumb and a forefinger of a hand 6 of the surgeon, the protrusions 24a and 24b are arranged on opposite sides of an interdigital web between the thumb and the forefinger, and the handle unit 24 is grasped such that a surface defining the recess 24c between the two protrusions 24a and 24b comes into contact with the interdigital web.

A material forming the shaft 2:3 and the handle unit 24 is not limited and may be metal, synthetic resin, a composite material including at least any one thereof, or the like. The synthetic resin includes thermoplastic resin, thermosetting resin, elastomer, and rubber. For example, the shaft 23 and the handle unit 24 can be each a molding of synthetic resin. The shaft 23 and the handle unit 24 can be joined by a freely-selected fixedly joining method such as adhesion using an adhesive or welding.

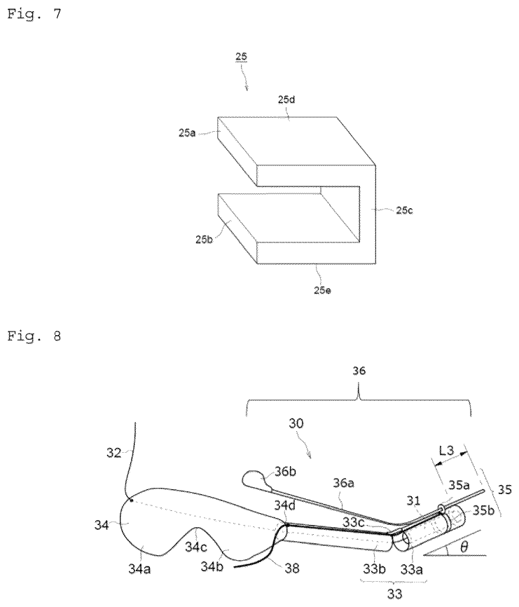

The endoscope assembly 20 further includes a mounting unit 25 that is attached to the distal shaft portion 23a of the shaft 23, to mount a treatment or surgery tool 26. In this embodiment, the mounting unit 25 is a holder unit, and, as illustrated in FIG. 7, is one member including two substantially parallel flat plate portions 25a and 25b and a vertical flat plate portion 25c that is continuous with the two substantially parallel flat plate portions 25a and 25b and extends substantially vertically with respect to the two substantially parallel flat plate portions 25a and 25b. Two parallel wide surfaces 25d and 25e opposite to surfaces facing each other in the two substantially parallel flat plate portions 25a and 25b form an outer surface of the mounting unit 25, and the treatment or surgery tool 26 is mounted onto one of the wide surfaces 25d and 25e while another one of the wide surfaces 25d and 25e is attached to the substantially cylindrical side surface of the shaft. In other words, one of the wide surfaces 25d and 25e of the mounting unit 25 holds the treatment or surgery tool 26, and another one of the wide surfaces 25d and 25e is fixedly attached to the substantially cylindrical side surface of the shaft. A material forming the mounting unit 25 is not limited and may be metal, synthetic resin, a composite material including at least any one thereof, or the like. The synthetic resin includes thermoplastic resin, thermosetting resin, elastomer, and rubber.

The treatment or surgery tool 26 includes a distal end portion 26a, an elongated portion 26b that mainly forms the tool and continuously extends from the distal end portion 26a at an angle to the distal end portion 26a, and an operating handle 26c attached to a proximal end of the elongated portion 26b. The operating handle 26c has a diameter larger than that of the elongated portion 26b for the operator (which may also be called a surgeon) to easily grasp. In this embodiment, the operating handle 26c has a wide surface formed into a shape of an inverted triangular plate with rounded corners for the operator to easily grasp with three fingers, but the operating handle 26c may have a wide surface formed into any other freely-selected shape that can be easily grasped by the operator with three fingers, such as a shape of a substantially semicircular plate. A material forming the operating handle 26c is not limited and may be metal, synthetic resin, a composite material including at least any one thereof, or the like. The synthetic resin includes thermoplastic resin, thermosetting resin, elastomer, and rubber.

The mounting unit 25 is positioned closer to the distal end of the shaft 23 than the handle unit 24. Further, in order that the handle unit 24 does not interfere with a movable range of the treatment or surgery tool 26 during an operation of the treatment or surgery tool 26 by the operator, it is preferred that the handle unit 24 extend outward along a radial direction of the shaft 23 from a position away from the mounting unit 25, specifically, a position at an angle of approximately 180° to the mounting unit 25 in the circumferential direction, on the circumference of the shaft 23.

Sizes of the respective components of the endoscope assembly 20 can be appropriately selected depending on applications. For example, for use in a transcanal endoscopic ear surgery, it is better for the shaft to have a smaller inner diameter, and the inner diameter is, for example, 2.7 mm or less. It is preferred that a length L1 of a portion on the distal side with respect to a spot mounted onto the mounting unit 25 in the treatment or surgery tool 26 be from approximately 5 mm to approximately 15 mm. It is preferred that a length L2 of a portion on the proximal side with respect to the spot mounted onto the mounting unit 25 in the treatment or surgery tool 26 be from approximately 70 mm to approximately 100 mm. It is preferred that a length from the distal end of the distal shaft portion 23a to a position at which the handle unit 24 is attached in the proximal shaft portion 23b be from approximately 50 mm to approximately 60 mm. It is preferred that a length of the handle unit 24 be from approximately 40 mm to 50 mm. The length L2 is set to a length larger than the length L1, thereby facilitating a fine operation with the distal end of the treatment or surgery tool 26 on “the principle of leverage”. The dimensions of the respective portions in a case in which the endoscope assembly 20 is used in a transcanal endoscopic ear surgery has been described above, but it is obvious that the operator can appropriately change the dimensions in the case of application to other than an ear surgery (surgery of other organs, and the like).

Next, operations of the endoscope assembly 20 are described. First, the treatment or surgery tool 26 is fixedly mounted onto the mounting unit 25 in advance. Subsequently, the operator grasps the proximal end portion of the shaft 23 of the endoscope assembly 20 and the operating handle 26c of the treatment or surgery tool 26, and inserts the treatment or surgery tool 26 and the shaft 23 into a body cavity of a subject from the distal ends. When the operator wants to change a position of a diseased part to be imaged, the operator operates the handle unit 24 with fingers (a forefinger, a middle finger, a ring finger, and the like) of the left hand and thus changes the position of the shaft 23, thereby being capable of changing the position of the diseased part to be imaged. In this embodiment, the distal shaft portion 23a of the shaft 23 is rotatable relative to the proximal shaft portion 23b, and the treatment or surgery tool 26 is fixedly mounted onto the mounting unit 25. Thus, when the surgeon rotates the operating handle 26c of the treatment or surgery tool 26 in a circumferential direction of the distal shaft portion 23a, only the distal shaft portion 23a is rotated in the circumferential direction independently of the proximal shaft portion 23b, which allows the treatment or surgery tool 26 to be freely moved to any position on the circumference about the longitudinal axis of the distal shaft portion 23a (and the shaft 23). Further, the operator can operate the treatment or surgery tool 26 separately from an operation of the handle unit 24. For example, by grasping and operating the operating handle 26c with a thumb, a forefinger, and a middle finger in the manner of holding chopsticks, or moving the operating handle 26c with only a forefinger, the operator can perform treatment such as aspiration, grasping, traction, dissection, and division with the distal end portion 26a of the treatment or surgery tool 26. Moreover, the operator can operate a second treatment or surgery tool different from the treatment or surgery tool 26 with the right hand and perform treatment, separately from an operation with the left hand. When the treatment of the patient ends, the operator withdraws the endoscope assembly 20 in a proximal-end direction while grasping the handle unit 24 between the thumb and the forefinger, and removes the endoscope assembly 20 from the body cavity of the subject.

Effects produced by the endoscope assembly 20 for operation assistance for a treatment or surgery tool according to the above-mentioned second embodiment are described.
The surgeon can operate the endoscope and the treatment or surgery tool with the same hand.
The surgeon can operate the treatment or surgery tool with a hand holding the endoscope and can operate another treatment or surgery tool with another hand, which enables a two-hand operation by the surgeon. Procedures such as aspiration, grasping, traction, dissection, and division can be performed with two hands. Alternatively, another operation can be also performed with two hands.
The shaft 23 includes two members of the distal shaft portion 23a and the proximal shaft portion 23b, and the mounting unit 25 is fixedly attached to the distal shaft portion 23a. Thus, by rotating the operating handle 26c of the treatment or surgery tool 26 in the circumferential direction of the distal shaft portion 23a, the surgeon can freely move the treatment or surgery tool 26 to any position on the circumference about the longitudinal axis of the distal shaft portion 23a (and the shaft 23).
The treatment or surgery tool 26 is designed so as to make it easy to perform a fine surgical operation using a leverage ratio between an operation of the operating handle unit 26c in hand and a distal end of the treatment or surgery tool 26.

The endoscope assembly 20 for operation assistance for a surgery tool according to the above-mentioned second embodiment can be modified as follows.
There may be provided a water flow mechanism that causes flow of a water stream from the proximal end to the distal end of the distal shaft portion 23a in a portion having a ring-shaped section between the distal shaft portion 23a and a cylindrical region accommodating the lenses and the optical fiber in the distal shaft portion 23a.

### Third Embodiment

Next, an endoscope assembly 30 for operation assistance for a treatment or surgery tool according to a third embodiment of the present invention is described. Description of members corresponding to those in the first embodiment of the present invention is omitted.

FIG. 8 is a perspective view of the endoscope assembly 30 for operation assistance for a treatment or surgery tool according to the third embodiment. The endoscope assembly 30 includes a CMOS image sensor 31 constituting an imaging element and a substantially cylindrical shaft 33, and the shaft 33 includes a distal shaft portion 33a that accommodates the CMOS image sensor 31 and is hollow and substantially cylindrical, and a proximal shaft portion 33b. The distal shaft portion 33a and the proximal shaft portion 33b are coupled by a flexible coupling member 33c provided therebetween, and the distal shaft portion 33a is rotatable relative to the proximal shaft portion 33b. A proximal end and a distal end of each of the distal shaft portion 33a and the proximal shaft portion 33b are sealed, and hence water is prevented from entering the inside of the distal shaft portion 33a and the proximal shaft portion 33b.

In the shaft 33, an optical fiber (not shown) extends along a longitudinal direction of the shaft 33, and illuminating light from a light source (not shown) such as an LED is guided from a proximal end to a distal end by the optical fiber (not shown) optically connected to the light source (not shown), to irradiate a subject. Such a configuration of an optical system for an endoscope is known.

At a distal end portion of the shaft 33, an object lens (not shown) is incorporated, and light from the subject passes through the object lens (not shown) from a light-transmitting distal end of the distal shaft portion 33a of the shaft 33, and forms an image in the CMOS image sensor 31. The image of the subject picked up in the CMS image sensor 31 is transmitted to a signal cable 32 and is subjected to signal processing. The signal cable 32 is connected to a computer, and the image of the subject can be observed with a display device connected to the computer.

The endoscope assembly 30 further includes a handle unit 34 that extends from the shaft 33 (and the proximal shaft portion 33b) while expanding. Specifically, the handle unit 34 extends outward along a radial direction of the shaft 33 (and the proximal shaft portion 33b) beyond a diameter of the shaft 33 (and the proximal shaft portion 33b), and extends beyond the proximal end of the shaft 33 (and the proximal shaft portion 33b). The handle unit 34 includes two protrusions 34a and 34b having curved surfaces, and a recess 34c is formed between the two protrusions 34a and 34b. Further, a substantially cylindrical hole 34d is provided at a distal end of the handle unit 34, and the proximal end portion of the proximal shaft portion 33b is fit into the hole 34d. The handle unit 34 has a continuously curved surface except a junction with the shaft 33 therein, and has a shape that is better fit for the surgeon's hand and reduces fatigue of the surgeon by its ergonomic design. Specifically, as illustrated in FIG. 9, during use of the endoscope assembly 30, the handle unit 34 is grasped by a thumb and a forefinger of the hand 6 of the surgeon, the protrusions 34a and 34b are arranged on opposite sides of an interdigital web between the thumb and the forefinger, and the handle unit 34 is grasped such that a surface defining the recess 34c between the two protrusions 34a and 34b comes into contact with the interdigital web.

A material forming the shaft 33 (that is, the distal shaft portion 33a, the proximal shaft portion 33b, and the coupling member 33c) and the handle unit 34 is not limited and may be metal, synthetic resin, a composite material including at least any one thereof, or the like. The synthetic resin includes thermoplastic resin, thermosetting resin, elastomer, and rubber. For example, the shaft 33 and the handle unit 34 can be each a molding of synthetic resin. The shaft 33 and the handle unit 34 can be joined by a freely-selected fixedly joining method such as adhesion using an adhesive or welding.

The endoscope assembly 30 further includes a mounting unit 35 that is attached to the distal shaft portion 33a of the shaft 33, to mount a treatment or surgery tool 36. In this embodiment, the mounting unit 35 includes a first loop 35a surrounding an elongated portion 36a of the treatment or surgery tool 36 at a predetermined position along a longitudinal direction of the elongated portion 36a, and a second loop 35b that is connected to the first loop 35a and surrounds the distal shaft portion 33a at a predetermined position along a longitudinal direction of the distal shaft portion 33a. In the mounting unit 35, the first loop 35a holds the treatment or surgery tool 36, and the second loop 35b is fixedly attached to the distal shaft portion 33a of the shaft 33.

The treatment or surgery tool 36, though confined by the first loop 35a, has a movable range wide enough for a distal end of the treatment or surgery tool 36 to draw a circle in a small range. A material forming the mounting unit 35 is not limited and may be metal, synthetic resin, a composite material including at least any one thereof, or the like. The synthetic resin includes thermoplastic resin, thermosetting resin, elastomer, and rubber.

The treatment or surgery tool 36 includes the elongated portion 36a and an operating handle 36b attached to a proximal end of the elongated portion 36a. The elongated portion 36a is made of a flexible material and is bendable. The operating handle 36b has a diameter larger than that of the elongated portion 36a for the operator to easily grasp. In this embodiment, the operating handle 36b has a wide surface formed into a shape of an inverted triangular plate with rounded corners for the operator to easily grasp with three fingers, but the operating handle 36b may have a wide surface formed into any other freely-selected shape that can be easily grasped by the operator with three fingers, such as a shape of a substantially semicircular plate. A material forming the operating handle 36b is not limited and may be metal, synthetic resin, a composite material including at least any one thereof, or the like. The synthetic resin includes thermoplastic resin, thermosetting resin, elastomer, and rubber.

The mounting unit 35 is positioned closer to the distal end of the shaft 33 (and the distal shaft portion 33a) than the handle unit 34. Further, in order that the handle unit 34 does not interfere with a movable range of the treatment or surgery tool 36 during an operation of the treatment or surgery tool 36 by the operator, it is preferred that the handle unit 34 extend outward along the radial direction of the shaft 33 from a position away from the mounting unit 35, specifically, a position at an angle of approximately 180° to the mounting unit 35 in the circumferential direction, on the circumference of the shaft 33.

Further, as illustrated in FIG. 9, the endoscope assembly 30 includes a string-shaped traction member 38. The traction member 38 has a distal end fixedly attached to the distal shaft portion 33a and has a proximal end that extends outward from the proximal shaft portion 33b and terminates on a proximal side with respect to the proximal end of the proximal shaft portion 33b. The traction member 38 is, for example, a wire. A part of the traction member 38 passes through the outside or bore of each of the distal shaft portion 33a and the proximal shaft portion 33b. The traction member 38 may extend through the outside of the distal shaft portion 33a and the outside of the proximal shaft portion 33b, may extend through the bore of the distal shaft portion 33a and the outside of the proximal shaft portion 33b, may extend through the outside of the distal shaft portion 33a and the bore of the proximal shaft portion 33b, or may extend through the bore of the distal shaft portion 33a and the bore of the proximal shaft portion 33b.

Sizes of the respective components of the endoscope assembly 30 can be appropriately selected depending on applications. For example, for use in a transcanal endoscopic ear surgery, it is better for the shaft to have a smaller inner diameter, and the inner diameter is, for example, 2.17 m or less. It is preferred that a length of a portion on a distal side with respect to a spot mounted onto the mounting unit 35 in the treatment or surgery tool 36 be from approximately 5 mm to approximately 15 mm. It is preferred that a length L3 from the distal end of the distal shaft portion 33a to the distal end of the treatment or surgery tool 36 be from approximately 5 mm to approximately 15 mm.

It is preferred that a length of a portion on a proximal side with respect to the spot mounted onto the mounting unit 35 in the treatment or surgery tool 36 be from approximately 70 mm to approximately 100 mm. It is preferred that a length from the distal end of the distal shaft portion 33a to a position at which the handle unit 34 is attached in the proximal shaft portion 33b be from approximately 50 mm to approximately 60 mm. It is preferred that a length of the handle unit 34 be from approximately 40 mm to 50 mm.

As illustrated in FIG. 9, an external ear canal length L4 is from approximately 25 mm to 30 mm, an external ear canal diameter L5 is from 5 mm to 9 mm, a length L6 is from 5 mm to 8 mm, and a length L7 is from 5 mm to 10 mm. In a case in which the endoscope assembly 30 is used in a transcanal endoscopic ear surgery, it is only required to design the endoscope assembly 30 so as to be adaptable to the above-mentioned anatomical structure.

Next, operations of the endoscope assembly 30 are described. First, the treatment or surgery tool 36 is fixedly mounted onto the mounting unit 35 in advance. Subsequently, the operator grasps the proximal end portion of the shaft 33 of the endoscope assembly 30 and the operating handle 36b of the treatment or surgery tool 36, and inserts the treatment or surgery tool 36 and the shaft 33 into a body cavity of a subject from the distal ends. When the operator wants to change a position of a diseased part to be imaged, the operator operates the handle unit 34 with fingers (a forefinger, a middle finger, a ring finger, and the like) of the left hand and thus changes the position of the shaft 33, thereby being capable of changing the position of the diseased part to be imaged. The treatment or surgery tool 36 is fixedly mounted onto the mounting unit 35. Thus, when the surgeon rotates the operating handle 36b of the treatment or surgery tool 36 in a circumferential direction about a longitudinal axis of the distal shaft portion 33a, the surgeon can move the treatment or surgery tool 36 in the circumferential direction about the longitudinal axis of the distal shaft portion 33a (and the shaft 33). Further, when the traction member 38 is withdrawn toward the proximal side in an arrow direction, the distal shaft portion 33a is bent at an angle θ to the proximal shaft portion 33b. Supposing that the angle θ=0° under a state in which the distal shaft portion 33a extends in a straight line with the proximal shaft portion 33b and is not bent, θ can take a value in a range of 0° or more and 120° or less, inclusive.

Further, the operator can operate the treatment or surgery tool 36 separately from an operation of the handle unit 34. For example, by grasping and operating the operating handle 36b with a thumb, a forefinger, and a middle finger in the manner of holding chopsticks, or moving the operating handle 36b with only a forefinger, the operator can perform treatment such as aspiration, grasping, traction, dissection, and division with the distal end portion of the treatment or surgery tool 36. Moreover, the operator can operate another treatment or surgery tool different from the treatment or surgery tool 36 with the right hand and perform treatment, separately from an operation with the left hand. When the treatment of the patient ends, the operator withdraws the endoscope assembly 30 in a proximal-end direction while grasping the handle unit 34 between a thumb and a forefinger, and removes the endoscope assembly 30 from the body cavity of the subject.

Effects produced by the endoscope assembly 30 for operation assistance for a treatment or surgery tool according to the above-mentioned third embodiment are described.
The surgeon can operate the endoscope and the treatment or surgery tool with the same hand.
The surgeon can operate the treatment or surgery tool with a hand holding the endoscope and can operate another treatment or surgery tool with another hand, which enables a two-hand operation by the surgeon. Procedures such as aspiration, grasping, traction, dissection, and division can be performed with two hands. Alternatively, another operation can be also performed with two hands.
The shaft 33 includes three members of the distal shaft portion 33a, the proximal shaft portion 33b, and the flexible coupling member 33c, and the mounting unit 35 is fixedly attached to the distal shaft portion 33a. Thus, the surgeon can freely move the treatment or surgery tool 36 in the circumferential direction about the longitudinal axis of the distal shaft portion 33a (and the shaft 33) by rotating the operating handle 36b of the treatment or surgery tool 36 in the circumferential direction of the distal shaft portion 33a.
The endoscope assembly 30 includes the traction member 38, the proximal end of the traction member 38 is fixedly attached to the distal shaft portion 33a, and the distal end extends outward from the proximal shaft portion 33b. Thus, when the traction member 38 is withdrawn toward the proximal side in the arrow direction, the distal shaft portion 33a can be bent at an angle θ to the proximal shaft portion 33b.
The treatment or surgery tool 36 is designed so as to make it easy to perform a fine surgical operation using a leverage ratio between an operation of the operating handle unit 36b in hand and the distal end of the treatment or surgery tool 36.

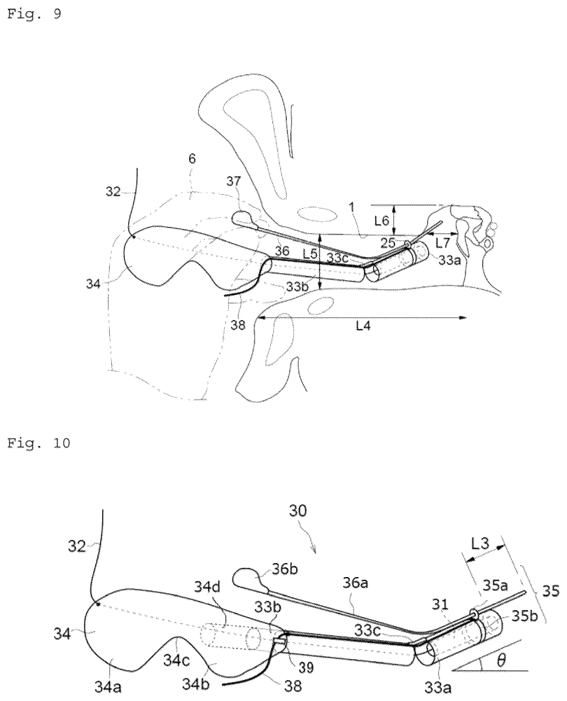

The endoscope assembly 30 for operation assistance for a treatment or surgery tool according to the above-mentioned third embodiment can be modified as follows.
As illustrated in FIG. 10, the hole 34d extends deep into the handle unit 34, and thus the proximal end portion of the proximal shaft portion 33b can move back and forth along an axial direction of the hole 34d in the hole 34d. With this configuration, the length of the proximal shaft portion 33b protruding from the handle unit 34, and in turn, the length of the shaft 33, can be adjusted, and, by rotation of the shaft 33 relative to the handle unit 34, a relative rotation angle between the handle and the shaft can be adjusted. Moreover, by providing a fixing member 39 that fixes the position of the proximal shaft portion 33b in the handle unit 34, it is possible to fix the position of the proximal shaft portion 33b relative to the handle unit 34, at a desired position. The fixing member 39 is provided on the distal side in the handle unit 34 in FIG. 10, and can move back and forth (that is, in the distal-end direction and in the proximal-end direction) along the axial direction of the hole 34d. More specifically, as illustrated in FIG. 11, the fixing member 39 includes an operating portion 39a exposed on the handle unit 34, a restricting portion 39b extending in the hole 34d, and a coupling portion 39c that passes through a hole (not shown) provided in the handle unit 34 and connects the operating portion 39a and the restricting portion 39b, and a section of the restricting portion 39b on the distal side is larger than that on the proximal side in the restricting portion 39b. Thus, when the fixing member 39 is moved in the distal-end direction, the restricting portion 39b of the fixing member 39 comes into contact with the proximal shaft portion 33b, to restrict movement of the proximal shaft portion 33b along the axial direction of the hole 34d. This enables fixing of the position of the proximal shaft portion 33b relative to the handle unit 34. When the fixing member 39 is moved in the proximal-end direction, the restricting portion 39b of the fixing member 39 moves away from the proximal shaft portion 33b, and the proximal shaft portion 33b is released from restriction.
As an alternative to the embodiment in which the fixing member 39 is provided as illustrated in FIG. 10 and FIG. 11, the position of the proximal shaft portion 33b relative to the handle unit 34 may be fixed at a desired position by fitting of one or more protruding portions provided in a portion (for example, a wall surface defining and forming the hole 34d) of the handle unit 34 in contact with the proximal shaft portion 33b, into one or more recessed portions provided in an outer peripheral surface of the proximal shaft portion 33b along the proximal shaft portion 33b. As long as movement of the proximal shaft portion 33b along the axial direction of the hole 34d can be restricted, the configuration of the fixing member is not limited.
In the third embodiment, the treatment or surgery tool 36 is described as an example of a treatment or surgery tool, but may be another treatment or surgery tool as described in the first embodiment.

### Fourth Embodiment

Next, an endoscope assembly 40 for operation assistance for a treatment or surgery tool according to a fourth embodiment of the present invention is described. Description of members corresponding to those in the second embodiment or the third embodiment of the present invention is omitted.

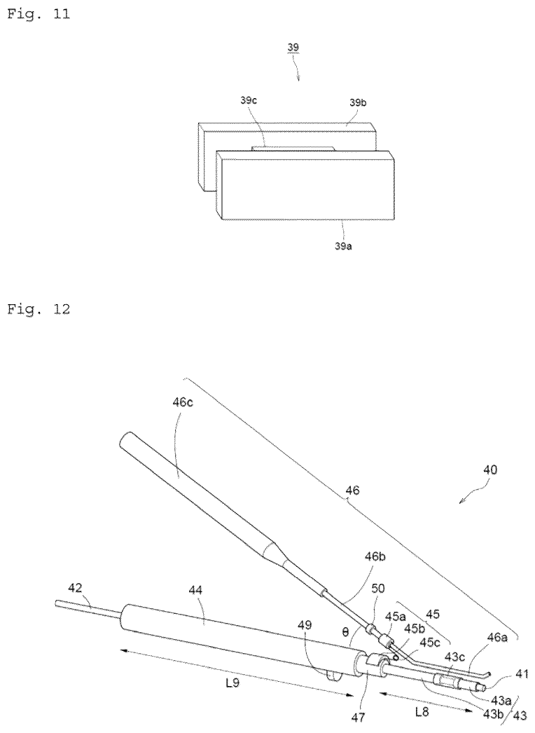

FIG. 12 is a perspective view of the endoscope assembly 40 for operation assistance for a treatment or surgery tool according to the fourth embodiment. The endoscope assembly 40 includes a CMOS image sensor 41 constituting an imaging element and a substantially cylindrical shaft 43. The shaft 43 includes a distal shaft portion 43a that accommodates the CMOS image sensor 41 and is hollow and substantially cylindrical, and a proximal shaft portion 43b. The distal shaft portion 43a and the proximal shaft portion 43b are coupled by a flexible coupling member 43c provided therebetween, and the distal shaft portion 43a and the proximal shaft portion 43b are rotatable relative to each other. In this embodiment, the flexible coupling member 43c is a silicone tube. An image of a subject picked up in the CMOS image sensor 41 is transmitted to a signal cable 42 connected to the CMOS image sensor 41 and is subjected to signal processing. The signal cable 42 is connected to a computer, and the image of the subject can be observed with a display device connected to the computer.

The endoscope assembly 40 further includes a handle unit 44 that extends from the shaft 43 while expanding. Specifically, the handle unit 44 extends outward along a radial direction of the proximal shaft portion 43b beyond a diameter of the proximal shaft portion 43b of the shaft 43. As illustrated in FIG. 13, a substantially tubular member 47 is provided on a part of an outer peripheral surface along a longitudinal direction of the proximal shaft portion 43b provided around the CMOS image sensor 41, and the handle unit 44 is provided on a part of an outer peripheral surface along a longitudinal direction of the substantially tubular member 47. The handle unit 44 has a length smaller than that of the proximal shaft portion 43b, and the handle unit 44 is positioned between both longitudinal ends of the proximal shaft portion 43b. The substantially tubular member 47 is rotatable about an axis in a circumferential direction, and is rotatable relative to the proximal shaft portion 43b. The proximal shaft portion 43b and the handle unit 44 are fixed to each other. As illustrated in FIG. 12, a length L8 from the distal shaft portion 43a to the substantially tubular member 47 can be appropriately changed depending on applications, but, for example, for use in a transcanal endoscopic ear surgery, the length L8 is from approximately 40 mm to 80 mm. A length L9 of the handle unit can also be appropriately changed depending on applications, but, for example, for use in a transcanal endoscopic ear surgery, the length L9 is from approximately 100 mm to 200 mm.

In this embodiment, the handle unit 44 is substantially cylindrical with a cross-sectional area that is substantially constant from end to end along a longitudinal direction, and has a compact shape that is less liable to interfere with an operation.

A material forming the shaft 43 and the handle unit 44 is not limited and may be metal, synthetic resin, a composite material including at least any one thereof, or the like. The synthetic resin includes thermoplastic resin, thermosetting resin, elastomer, and rubber. For example, the shaft 43 and the handle unit 44 can be each a molding of synthetic resin. The shaft 43 and the handle unit 44 can be joined by a freely-selected fixedly joining method such as adhesion using an adhesive or welding.

The endoscope assembly 40 further includes a mounting unit 45 that is attached indirectly to the distal shaft portion 43a of the shaft 43 via the substantially tubular member 47, to mount a treatment or surgery tool 46. The mounting unit 45 includes a first fixing portion 45a for fixing the mounting unit 45 to the treatment or surgery tool 46, a second fixing portion 45b for fixing the mounting unit 45 to a side surface of the substantially tubular member 47, and a coupling portion 45c coupling the first fixing portion 45a and the second fixing portion 45b. In this embodiment, the coupling portion 45c is illustrated as a torsion spring, but may be a leaf spring or a freely-selected elastic member that is deformable by biasing. The coupling portion 45c is substantially V-shaped with one end thereof sandwiched between the first fixing portion 45a and an elongated portion 46b of the treatment or surgery tool 46, and with another end thereof sandwiched between the second fixing portion 45b and the substantially tubular member 47. An angle Θ of the V-shape can be changed by movement of the position of the treatment or surgery tool 46 relative to the handle unit 44. For example, with no bias applied, Θ has a value of approximately 30 degrees, and it is possible to reduce this angle by bringing the treatment or surgery tool 46 close to the handle unit 44. The mounting unit 45 is positioned closer to the distal end of the shaft 43 than the handle unit 44. On the proximal side with respect to a position at which the mounting unit 45 is fixed in the treatment or surgery tool 46, a tubular member 50 for fixing the treatment or surgery tool 46 on the proximal side with respect to the mounting unit 45 is provided in such a manner as to be fixed to the elongated portion 46b of the treatment or surgery tool 46 or the first fixing portion 45a so that the treatment or surgery tool 46 can be inserted and fit into an opening of the tubular member 50. In a case in which the tubular member 50 is fixed to the elongated portion 46b of the treatment or surgery tool 46, the tubular member 50 is removable or slidable relative to the elongated portion 46b, and may be fixable at a freely-selected position along a longitudinal direction of the elongated portion 46b.

The treatment or surgery tool 46 includes a distal end portion 46a, the elongated portion 46b continuously extending from the distal end portion 46a at an angle to the distal end portion 46a, and an operating handle 46c attached onto the elongated portion 46b on the proximal side of the elongated portion 46b. The operating handle 46c has a diameter larger than that of the elongated portion 46b for the operator to easily grasp. A material forming the operating handle 46c is not limited and may be metal, synthetic resin, a composite material including at least any one thereof, or the like. The synthetic resin includes thermoplastic resin, thermosetting resin, elastomer, and rubber. In this embodiment, the treatment or surgery tool 46 is connected to the substantially tubular member 47 via the mounting unit 45. Thus, by rotating the substantially tubular member 47, it is possible to rotate the position of the treatment or surgery tool 46 about an axis of the substantially tubular member 47 (that is also an axis of the shaft 43) without rotating the proximal shaft portion 43b of the shaft 43.

Further, as illustrated in FIG. 13, the endoscope assembly 40 includes a string-shaped traction member 48. In this embodiment, the traction member 48 has a distal end fixedly attached to the distal shaft portion 43a, passes between the CMOS image sensor 41 and the distal shaft portion 43a and between the CMOS image sensor 41 and the proximal shaft portion 43b, and extends outward from the proximal shaft portion 43b via a hole or a slit provided in a side surface of the proximal shaft portion 43b on the proximal side with respect to a proximal end of the substantially cylindrical member 47. The traction member 48 is, for example, a wire. The distal end of the traction member 48 is fixedly attached to the distal shaft portion 43a, the traction member 48 extends through the bore of the distal shaft portion 43a and the bore of the proximal shaft portion 43b, and a proximal end of the traction member 48 terminates in the inside of the handle unit 44. Alternatively, the traction member 48 may extend through the outside of the distal shaft portion 43a, the proximal shaft portion 43b, and the outside of the substantially cylindrical member 47. The traction member 48 may extend through the bore of the distal shaft portion 43a, the outside of the proximal shaft portion 43b, and the bore or outside of the substantially cylindrical member 47, or may extend through the outside of the distal shaft portion 43a and the bore of the proximal shaft portion 43b.

A grasping portion 49 that can slide so as to selectively advance or withdraw the traction member 48 extends through the handle unit 44 from the outside of the handle unit 44 to the inside of the handle unit 44. The grasping portion 49 can be also referred to as a tab or a knob, and can have a freely-selected shape such as a pillar shape or a disc shape. The grasping portion 49 can move along a longitudinal direction (a direction indicated by an arrow in FIG. 13) of the handle unit 44. The proximal end of the traction member 48 is fixed to the grasping portion 49, and, when the grasping portion 49 is moved toward the distal end of the handle unit 44, the traction member 48 is advanced out of the handle unit 44, which allows the distal shaft portion 43a to be moved farther toward the distal side. When the grasping portion 49 is moved toward the proximal end of the handle unit 44, the traction member 48 is withdrawn to the inside of the handle unit 44, which allows the distal shaft portion 43a to be withdrawn farther toward the proximal side. A mechanism for selectively advancing and withdrawing the traction member 48 through an external operation of the handle unit 44 is not limited to the grasping portion 49 having a sliding configuration, and can have various configurations of members or devices for reeling up the traction member 48.

Next, operations of the endoscope assembly 40 are described. First, the treatment or surgery tool 46 is fixedly mounted onto the mounting unit 45 in advance. In this embodiment, owing to the substantially tubular shape of the first fixing portion 45a, the treatment or surgery tool 46 can be inserted into and/or removed from an opening of the first fixing portion 45a. This enables replacement of the treatment or surgery tool 46 to be mounted onto the mounting unit 45.

Subsequently, the operator grasps the handle unit 44 of the endoscope assembly 40 and the operating handle 46c of the treatment or surgery tool 46, and inserts the treatment or surgery tool 46 and the shaft 43 into a body cavity of a subject from the distal ends. When the operator wants to change a position of a diseased part to be imaged, the operator grasps the grasping portion 49 with fingers (a forefinger, a middle finger, a ring finger, and the like) of one hand in the manner of holding chopsticks and thus changes the position of the shaft 43, thereby being capable of changing the position of the diseased part to be imaged. The treatment or surgery tool 46 and the substantially tubular member 47 are fixedly mounted onto the mounting unit 45, and the surgeon can rotate the operating handle 46c of the treatment or surgery tool 46 in a circumferential direction about the longitudinal axis of the substantially tubular member 47, to move the treatment or surgery tool 46 in the circumferential direction about the longitudinal axis of the substantially tubular member 47. Further, by withdrawing the traction member 48 through an operation of the grasping portion 49, it is possible to bend the distal shaft portion 43a at an angle to the proximal shaft portion 43b.

Further, the operator can operate the treatment or surgery tool 46 separately from an operation of the handle unit 44. For example, the operator can perform treatment such as aspiration, grasping, traction, dissection, and division with the distal end portion of the treatment or surgery tool 46 through an operation of the operating handle 46c. Moreover, the operator can operate another treatment or surgery tool different from the treatment or surgery tool 46 with the right hand and perform treatment, separately from an operation with the left hand. When the treatment of the patient ends, the operator withdraws the endoscope assembly 40 in the proximal-end direction while grasping the handle unit 44 between a thumb and a forefinger, and removes the endoscope assembly 40 from the body cavity of the subject.

Effects produced by the endoscope assembly 40 for operation assistance for a treatment or surgery tool according to the above-mentioned fourth embodiment are described.
The surgeon can operate the endoscope and the treatment or surgery tool with the same hand.
The surgeon can operate the treatment or surgery tool with a hand holding the endoscope and can operate another treatment or surgery tool with another hand, which enables a two-hand operation by the surgeon. Procedures such as aspiration, grasping, traction, dissection, and division can be performed with two hands. Alternatively, another operation can be also performed with two hands.
With the use of an elastic member for the mounting unit 45, it is possible to change the position of the treatment or surgery tool 46 relative to the handle unit 44 by biasing the elastic member through an operation of the treatment or surgery tool 46.
The mounting unit 45 is fixedly attached to the substantially tubular member 47. Thus, when the surgeon rotates the operating handle 46c of the treatment or surgery tool 46 in the circumferential direction of the substantially tubular member 47, the surgeon can freely move the treatment or surgery tool 46 in the circumferential direction about the longitudinal axis of the substantially tubular member 47 (and the shaft 43) without rotating the proximal shaft portion 43b of the shaft 43.
The endoscope assembly 40 includes the traction member 48, the distal end of the traction member 48 is fixedly attached to the distal shaft portion 43a, and the proximal end of the traction member 48 terminates at the slidable grasping portion 49. Thus, by moving the grasping portion 49 to withdraw the traction member 48 toward the proximal side, it is possible to bend the distal shaft portion 43a at an angle to the proximal shaft portion 43b.

The present invention has been described with reference to the first to fourth embodiments, but, for example, a member in a certain embodiment can be applied to another embodiment.

For example, the mounting unit 35 including the first loop 35a and the second loop 35b in the third embodiment may be used for surrounding the respective peripheries of the elongated portion 26b of the treatment or surgery tool 26 and the distal shaft portion 23a in the second embodiment.

For example, the hole 34d of the handle unit 34, the proximal shaft portion 33b capable of moving in the hole 34d, and further the fixing member 39, which are illustrated in FIG. 10, may be applied to the endoscope assembly 20 in the second embodiment. That is, the proximal shaft portion 23b is made movable in a hole provided in the handle unit 24, and the proximal shaft portion 23b, and in turn, the shaft 23 are caused to slide relative to the handle unit 24, so that a distance between the handle unit 24 and the distal end of the shaft 23 can be adjusted.

### EXAMPLES

### Example 1: Example of Transmastoid Superior Semicircular Canal Closure Surgery

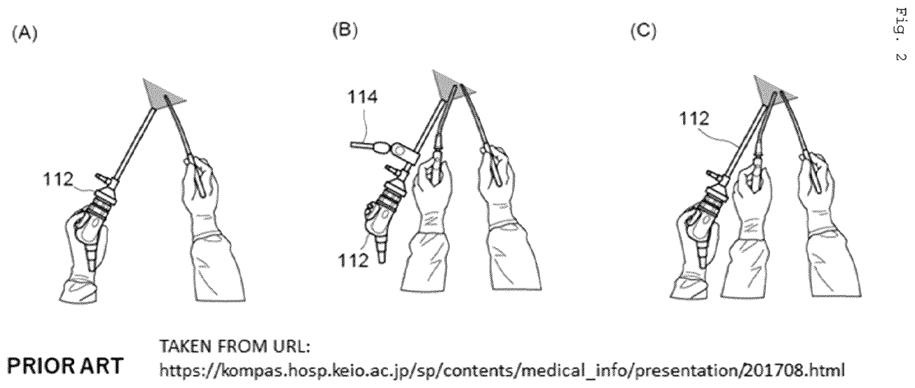

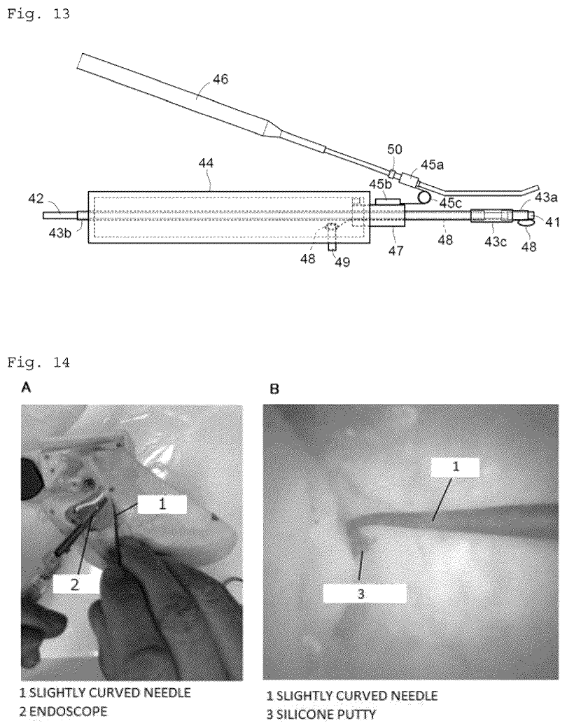

In order to verify whether or not the endoscope assembly for operation assistance for a treatment or surgery tool can effectively improve operability of a surgery, a model surgery close to a transmastoid superior semicircular canal closure surgery was performed using a temporal bone model. To this end, in a one-hand surgery (FIG. 2(A), FIG. 14(A), and FIG. 14(B)) and a surgical procedure using a prototype endoscope according to the fourth embodiment of the present invention, autologous tissues (imitated by blue silicone putty) were introduced, and a time taken to close a superior semicircular canal was measured. Then, respective results were compared (Table 1 and Table 2). The temporal bone model used in the present experiment was a Phacon bone, “Schmidt”, left ear (NB503) manufactured by PHACON GmbH.

### Method of Experiment

The temporal bone model was subjected to mastoidectomy in advance, the superior semicircular canal was opened from the inner side surface thereof, and water was stored in a mastoid antrum. A model surgery was performed under the following two conditions, to evaluate the endoscope assembly for operation assistance for a treatment or surgery tool.

(1) One-Hand Surgery The endoscope was held by a left hand, and a slightly curved needle was held by a right hand. A small piece of silicone putty wad put to a distal end and introduced into the superior semicircular canal, and an open cavity was closed at front and rear ends (FIG. 2(A), FIG. 14(A), and FIG. 14(B)).

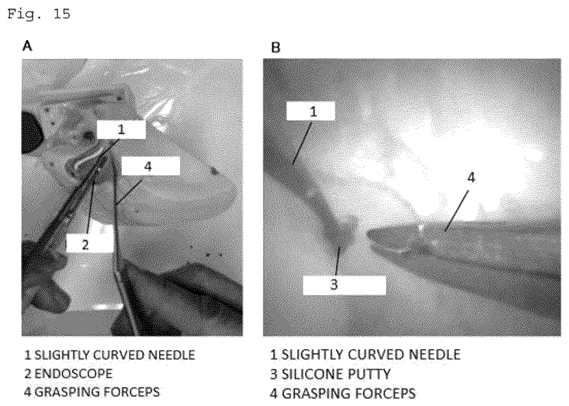

(2) While the prototype endoscope according to the fourth embodiment of the present invention was held by a left hand, the surgery tool 46 was operated by left-hand fingers, and at the same time, the slightly curved needle held by a right hand was operated. A small piece of silicone putty was put and introduced into the superior semicircular canal. Then, an open cavity was closed at front and rear ends (FIG. 15(A) and FIG. 15(B)).

The results are shown in Tables 1 and 2. In the method (2) using the prototype endoscope according to the fourth embodiment, the surgery tool 46 held down the small piece of the silicone putty so as not to hinder the small piece of silicone putty from being introduced due to adhesion of the small piece of the silicone putty to the slightly curved needle, which cooperatively facilitates the introduction, to thereby significantly shorten an operating time. An average time in the cases in which the endoscope assembly of the example was used was 56.542 seconds (standard deviation of 8.59), and an average time in the cases of a one-hand surgery was 84.164 seconds (standard deviation of 15.0). (P=0.007, Student's t-test)

TABLE 1
CASE OF USE OF CHOPSTICKS-LIKE ENDOSCOPE

| | |
|---|---|
| FIRST TIME | 61.47 |
| SECOND TIME | 69.06 |
| THIRD TIME | 53.40 |
| FOURTH TIME | 50.41 |
| FIFTH TIME | 48.37 |
| AVERAGE | 56.54 |

TABLE 2
CASE OF ONE-HAND SURGERY

| | TIME (s) |
|---|---|
| FIRST TIME | 94.84 |
| SECOND TIME | 71.12 |
| THIRD TIME | 83.57 |
| FOURTH TIME | 68.18 |
| FIFTH TIME | 103.11 |
| AVERAGE | 84.16 |

### INDUSTRIAL APPLICABILITY

The endoscope assembly of the present invention can be used in various surgeries using an endoscope. The endoscope assembly is mainly intended for use in a transcanal endoscopic ear surgery (including a transmastoid underwater endoscopic ear surgery), but is applicable also to a transcanal endoscopic ear surgery on condition that a requirement about a sufficient external ear canal length or the like is satisfied. For example, implantation of a piston or a columella in a stapes surgery or tympanoplasty is facilitated. Further, the endoscope assemblies of the second and third embodiments include the bendable shafts 23 and 33, and thus are applicable also to extirpation of cholesteatoma developing in a direction of an epitympanum or a mastoid antrum. Further, the endoscope assemblies are applied to all types of underwater endoscopic internal ear surgeries (such as a semicircular canal fistula, a superior semicircular canal dehiscene syndrome, a cochlear implant surgery, and DDS).

Besides, the endoscope assembly of the present invention is applied to not only an ear surgery, but also an endoscopic nasal/paranasal cavity surgery, a transnasal-endoscopic anterior skull base surgery, an endoscopic pharyngeal/laryngeal surgery, an endoscopic neurosurgery, an endoscopic obstetric/gynecologic surgery, an arthroscopic surgery, a cystoscopic surgery, and a so-called keyhole surgery in an endoscopic surgery in the field of veterinary medicine, and improvements in operability and safety thereof can be expected.

### EXPLANATION OF REFERENCE NUMERALS

10, 20, 30, 40 endoscope assembly, 11 CMOS image sensor serving as imaging element, 13, 23, 33, 43 shaft, 14, 24, 34, 44 handle unit, 15, 25, 35, 45 mounting unit, 16, 26, 36, 46 treatment or surgery tool, 21 CCD camera serving as imaging element, 16d, 26c, 36b operating handle, 23a, 33a, 43a distal shaft portion, 23b, 33b, 43b proximal shaft portion, 33c, 43c coupling member, 38, 48 traction member, 39 fixing member, 47 substantially tubular member, 49 grasping portion

1. An endoscope assembly for operation assistance for a treatment or surgery tool, the endoscope assembly comprising:
an imaging element;
a shaft, which is configured to accommodate the imaging element, or is connected to the imaging element;
a handle unit extending from the shaft while expanding; and
a mounting unit attached to the shaft, to mount a treatment or surgery tool.

2. The endoscope assembly according to claim 1, wherein the mounting unit is positioned closer to a distal end of the shaft than the handle unit.

3. The endoscope assembly according to claim 1, wherein the treatment or surgery tool is mounted onto the mounting unit.

4. The endoscope assembly according to claim 3, wherein the treatment or surgery tool is a stick-shaped tool including an operating handle.

5. The endoscope assembly according to claim 1,
wherein the shaft includes a distal shaft portion and a proximal shaft portion, and
wherein the distal shaft portion is rotatable relative to the proximal shaft portion.

6. The endoscope assembly according to claim 5, wherein the distal shaft portion and the proximal shaft portion are coupled by a flexible coupling member.

7. The endoscope assembly according to claim 5, further comprising a traction member that has a proximal end fixedly attached to the distal shaft portion, and has a distal end extending outward from the proximal shaft portion,
wherein the distal shaft portion is bent when the traction member is withdrawn toward a proximal side.

8. The endoscope assembly according to claim 1,
wherein the shaft includes a distal shaft portion, a proximal shaft portion, and a substantially tubular member that is attached to an outer peripheral surface of the proximal shaft portion and is rotatable relative to the proximal shaft portion, and
wherein the mounting unit for the treatment or surgery tool is attached to the substantially tubular member.

9. The endoscope assembly according to claim 1,
wherein the shaft includes a distal shaft portion and a proximal shaft portion,
wherein the handle unit is attached onto an outer periphery of the proximal shaft portion along a longitudinal part of the proximal shaft portion,
wherein the endoscope assembly further comprises:
a traction member that has a proximal end fixedly attached to the distal shaft portion, extends through an outside or a bore of each of the distal shaft portion and the proximal shaft portion, and terminates on a proximal side with respect to the proximal shaft portion; and
a mechanism extending through the handle unit from an outside of the handle unit to an inside of the handle unit, the mechanism being configured to selectively advance or withdraw the traction member.

10. The endoscope assembly according to claim 1, wherein the imaging element is accommodated in the shaft.

11. The endoscope assembly according to claim 1, wherein the imaging element is connected to a proximal end of the shaft.

12. An operation assisting device for a treatment or surgery tool, which is configured to be used in connection to an endoscope including an imagine device, the operation assisting device comprising:
a shaft configured to permit the imaging device to pick up an image of a subject via a distal end of the shaft,
wherein the shaft includes:
a handle unit extending from the shaft while expanding; and
a mounting unit attached to the shaft, to mount a treatment or surgery tool.
